(19) United States
(12) Patent Application Publication
(10) Pub. No.: US 2008/0311012 A1
(43) Pub. Date: Dec. 18, 2008

(54) Transmissive window for hydrooptic disinfection system

(76) Inventor: Uri Levy, Rehovot (IL)

Correspondence Address: Pearl Cohen Zedek Latzer, LLP, 1500 Broadway, 12th Floor, New York, NY, 10036, US

(21) Appl. No.: 11/808,628

(22) Filed: Jun. 12, 2007

(51) Int. Cl. A61L 2/10; A61L 2/02

(52) U.S. Cl. 422/186.3; 422/129

(57) ABSTRACT

Some demonstrative embodiments of the invention include an illumination-based liquid disinfection device. The disinfection device may include, for example, a conduit to carry a flowing liquid to be disinfected, the conduit having an inlet to receive the liquid and an outlet to discharge the liquid; at least one illumination source external to the conduit to illuminate the liquid with light through a shaped non-flat window. The non-flat window may redistribute the light in the conduit so as to improve uniformity of light-dose received by entities such as micro-organisms and or chemicals within the liquid.

### BACKGROUND OF THE INVENTION

Ultraviolet liquid disinfection systems using UV light source located within the chamber through which the liquid flows have been long known. Other UV disinfection systems use an external light source to irradiate the liquid through a window made of a material transparent to the radiation.

The irradiation of the liquid inactivates microorganisms in the liquid, if the irradiation intensity and exposure duration are above a minimum dose level (often measured in units of miliJoules per square centimeter). Further, higher irradiation intensity would cause higher level of inactivation of the microorganisms within the chamber. Ideally, UV-based disinfection systems should be constructed such that each microbe that crosses the chamber in any possible stream line is irradiated with the same UV dose.

Conventional disinfection systems utilize one or more UV radiation source immersed in the liquid within the reactor. Other disinfection systems include an external UV radiation source that radiates the liquid via a transmissive window. The window may be located proximate to an entrance opening of the liquid. For such a configuration, it has been found that the flow velocity of liquid within the area located near the center of the window is relatively small. This area, however, has a relatively high intensity radiation due to its location in proximity to the UV source. This combination of small velocities and high UV-irradiation intensity, leads to undesired condition of unequal delivery of UV dose to microorganisms in the system. A solution to this problem is highly desired.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features and advantages thereof, may best be understood by reference to the following detailed description when read with the accompanied drawings in which:

FIG. 1 is a conceptual illustration of an illumination-based disinfector according to some demonstrative embodiments of the invention;

FIG. 2 is a conceptual illustration of an illumination-based disinfector with two illumination sources according to some demonstrative embodiments of the invention;

FIG. 3 depicts exemplary illustrations of an optical window in accordance with embodiments of the invention; and

FIG. 4 is an illustration of a shaped optical window according to some demonstrative embodiments of the invention.

It will be appreciated that for simplicity and clarity of illustration, elements shown in the drawings have not necessarily been drawn accurately or to scale. For example, the dimensions of some of the elements may be exaggerated relative to other elements for clarity. Further, where considered appropriate, reference numerals may be repeated among the drawings to indicate corresponding or analogous elements. Moreover, some of the blocks depicted in the drawings may be combined into a single function.

### DETAILED DESCRIPTION OF EMBODIMENTS OF THE INVENTION

In the following detailed description, numerous specific details are set forth in order to provide a thorough understanding of the invention. However, it will be understood by those of ordinary skill in the art that the present invention may be practiced without these specific details. In other instances, well-known methods, procedures, components and circuits may not have been described in detail so as not to obscure the present invention.

Some demonstrative embodiments of the invention include a device to illuminate a medium having entities suspended therein, e.g., to disinfect the medium, as described in detail below. It will be appreciated that the term “medium” as used herein may refer to any liquid, e.g., including water or water-based liquid, which may be, for example, intended to be disinfected.

It will be appreciated that the term “entity” as used herein may refer to any organism, bacteria, microorganism, being, creature, microbe, germ, virus, organic contaminator, non-organic contaminator, oxidizable toxic or contaminator; any cumulative noxious species of biological or chemical origin; any oxidizing particle, fragment or element, e.g., Hydrogen peroxide or Titanium dioxide, intended to oxidize a contaminator; and/or the like. It will be appreciated that the phrase “entities suspended in a medium” as used herein may refer to any entity which may be suspended, contained, or mixed in the medium; and/or carried by the medium.

In some demonstrative embodiments of the invention, the device may include a conduit, for example, a reactor, a vessel, a chamber, e.g., an elongated chamber, to carry the medium. The conduit may have an inlet to receive the medium and an outlet to discharge the medium. The device may also include at least one illumination source to illuminate the conduit with light having a spatial non-uniform light flux distribution, also known as light intensity distribution.

In some demonstrative embodiments of the invention, each entity in the medium may accumulate a certain light dose, for example, ultraviolet dose, e.g., in units of energy/area. It will be appreciated that the phrase “light dose” or “dose” as used herein may refer to a determined, computed, calculated, simulated, modeled, estimated, anticipated, assessed, assigned and/or planned amount of light radiation.

In some demonstrative embodiments of the invention, the device may affect at least part, e.g., substantially most or even all of the entities suspended in the medium. In some embodiments of the invention, the device may activate most or even all of oxidizing particles suspended within the medium. The probability of entities affecting, also referred to herein as “kill probability”, may be related to the accumulated light dose. Higher light dose would usually cause higher kill probability.

Some demonstrative embodiments of the invention, e.g., as described below, may refer to using Ultra-Violet (UV) light to illuminate the medium, e.g., to disinfect the medium, and/or to oxidize the particles within the medium. However, it will be appreciated by those skilled in the art, that in other embodiments of the invention, light of any other suitable spectrum may be used.

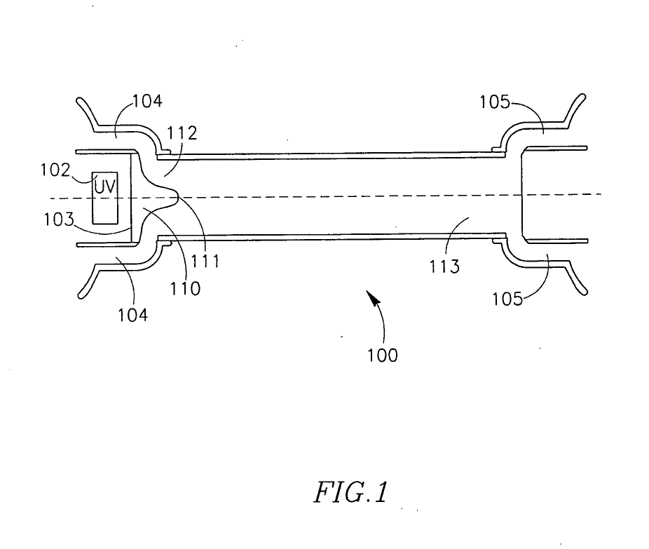

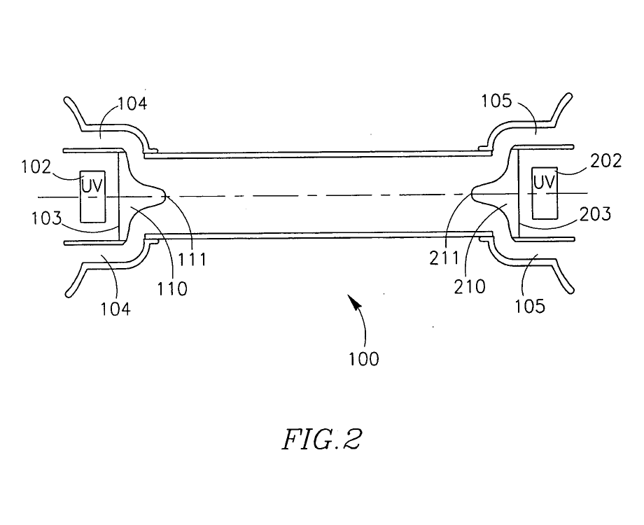

Reference is now made to FIG. 1, which conceptually illustrates an illumination-based disinfector 100 according to some demonstrative embodiments of the invention. Reference is additionally made to FIG. 2, which conceptually illustrates an illumination-based disinfector 200 according to other demonstrative embodiments of the invention

According to some demonstrative embodiments of the invention, disinfector 100 may include a conduit 101 to carry a flowing medium to be disinfected and an external illumination source 102 to illuminate the medium within conduit 101. As depicted in FIG. 2, according to another embodiment of the present invention, disinfector 200 may comprise two illumination sources 102 and 202, each at one end of conduit 201.

Although the invention is not limited in this respect, illumination source 102 may generate UV light of a suitable UV spectrum. For example, illumination source may include one or more UV lamps, e.g., a low-pressure UV, a medium-pressure UV lamp and/or a microwave-excited UV lamp, as are all known in the art.

Conduit 101 may have an inlet 104 to receive the medium, and an outlet 105 to discharge the medium. Conduit 101 may further include walls 106 may be made of transparent material, such as quartz, and a transparent window 103 located at the end of conduit 101 proximate to illumination source 102. Disinfector 100 may also include one or more external illumination sources 102 to illuminate conduit 101 with light having a spatial light intensity distribution. The light produced by illumination source 102 may be directed toward the medium within conduit 101 via window 103. In some embodiments of the invention, window 103 is made of material transparent to UV light, such as quartz. Any other suitable light-transparent material may be used. The conduit may be located inside a protective metal sleeve with an air gap between the conduit and the sleeve (not shown).

According to some embodiments of the invention, a reflector (not shown) may partially surround illumination source 102. The reflector may be positioned such that radiation from illumination source 102 may be reflected from the reflector in the direction of window 103. The reflector is designed to deflect light into conduit 101 through window 103 in a way that maximizes the UV output from illumination source 102.

In the exemplary illustration of FIG. 1 the window-lamp assembly is illustrated as being in proximity to the water inlet. It should, however, be understood to a person skilled in the art that embodiments of the invention are not limited in this respect and alternatively or additionally, the window-lamp assembly may be positioned in proximity to the outlet of the conduit. As depicted in FIG. 2, according to another embodiment of the present invention, disinfector 200 may comprise two windows 103 and 203, each at one end of conduit 201.

According to some demonstrative embodiments of the invention, window 103 may be configured in a tapered, non-planar figure. As demonstrated in FIG. 1, Window 103 may include a base 110 having an external planar surface and a non-planar internal surface extending into the inner space of the conduit. As schematically illustrated at FIGS. 1 and 2, the window narrows towards the inner space of the conduit. The overall figure of the window may be of a cone-like, a pyramid-like or a 3-D Gaussian-like. Any other symmetrical or unsymmetrical shape or figure having its base wider than its top may be used. Window 103 may include a tip 111, which may be pointed, sharpened, truncated, cut or may have any other geometry. Window 103 may reduce the flow stagnation zone by better distributing the light entering the water.

Although, embodiments of the present invention are not limited in this respect, it is estimated that during the UV disinfection operation, the tapered figure of the window may improve the efficiency of UV disinfection and increase kill probability, namely the probability to inactivate the entities being in the medium flowing in conduit 101.

According to some demonstrative embodiments of the invention, the exact shape of window 103 may be pre determined based on several factors, such as for example, the location and shapes of inlet 104, the flow velocity distribution of the medium to be disinfected, the radiation distribution, the degree of clarity of the medium being disinfected, the type of glass of which the window is made, the type of glass of which the chamber is made or any other factor.

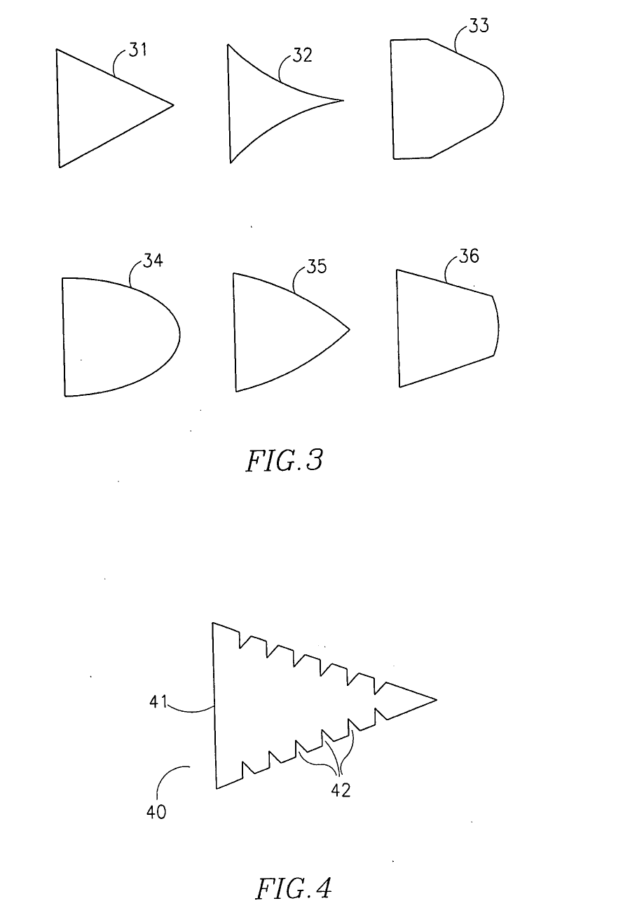

Reference is now made to FIG. 3, which depicts cross sectional views of exemplary optical windows in various shapes in accordance with embodiments of the invention. It should be understood that these specific exemplary configurations of window 103 do not limit the scope of the invention and other configurations are likewise applicable.

The side cross section of window 103 or 203, may have a substantially tapered shape where the window includes a tip and a base, the tip being narrower than the base, for example, the cross section of window 103 may be substantially pyramidal-like, 3D-Gaussian like, or any general non-flat cylindrically symmetrical or non-symmetrical suitable shape. The shape of the window may be, for example, that of a frustum or conical frustum, or a portion of a sphere, ellipse or other rounded shape. It should be understood to a person skilled in the art that embodiments of the invention are not limited in this respect and other suitable shapes may be used. According to some embodiments of the invention, the shape of the inlet window, e.g., window 103, and the shape of the outlet window, e.g., window 203 may be different.

The shape of window may affect the refraction of light and accordingly may change the light flux distribution. Additionally, it may modify the flow-pattern of the medium to be disinfected. While the exemplary illustrated cross sectional views of the windows are symmetrical with respect to a longitudinal axis, it should be understood to a person skilled in the art that embodiments of the invention are not limited in this respect and according to other embodiments of the present invention the window may be unsymmetrical with respect to the longitudinal axis. The specific shape of the window may be pre-designed based on several factors, as discussed above.

FIG. 4 is an illustration of a shaped optical window according to some demonstrative embodiments of the invention. According to embodiments of the present invention, the transmissive window of conduit 101, 201 may have local surface features on its surface. Although the invention is not limited in this respect, an exemplary window 40 having a cross section of a triangle is shown. Accordingly, the shape of window 40 may be, for example, a cone or a triangular pyramid. It should be understood that the basic shape of the shaped window according to other embodiments of the present invention may have any general non flat shape and the set of shapes depicted in FIG. 3 are only illustrative. According to embodiments of the present invention, the smooth surface of the transmissive window may be modified by pre-defined surface features 42 in order to further modify and control light distribution in the medium. For example, window 40 may include ridges, openings, bumps, slits, slots or saw-teeth. Any other suitable surface topography may be used.

According to some demonstrative embodiments of the invention, light from illumination source 102 may pass through window 40 into conduit 101. Window 40 may guide, direct or lead the light such that the light may be scattered, reflected or spread in accordance with both the basic shape of the window, e.g., the tapered shape of window 40, and the local features 42. The surface topography or modifications may improve the dose distribution function, for example, by allowing areas of high values of crossing velocities to receive higher light intensities and to improve the uniformity of the UV-dose delivered to the crossing microorganisms crossing the reactor along a stream line. Any suitable local features or shape modifications to control the manner of the light propagation and modify its distribution in the water may be used.

While certain features of the invention have been illustrated and described herein, many modifications, substitutions, changes, and equivalents may occur to those of ordinary skill in the art. It is, therefore, to be understood that the appended claims are intended to cover all such modifications and changes as fall within the true spirit of the invention.

What is claimed is:

1. An apparatus for disinfection a liquid, the apparatus comprising:
a conduit to carry a flowing liquid to be disinfected, said conduit having transparent walls, an inlet to receive said liquid, an outlet to discharge said liquid and at least one window, said window having a tapered figure; and
at least one illumination source external to said conduit to illuminate said liquid with light, said light propagates through said window.

2. The apparatus of claim 1, wherein the tapered figure is substantially conical, pyramidal or three-dimensional Gaussian.

3. The apparatus of claim 1, wherein the window comprises a non-planar surface extending into an inner space of said conduit.

4. The apparatus of claim 1, wherein the window is made of quartz.

5. The apparatus of claim 3, wherein the surface comprises pre-defined surface features.

6. The apparatus of claim 5, wherein the pre-defined surface features comprise ridges.

7. The apparatus of claim 1, wherein said at least one illumination source comprises two illumination sources.

8. The apparatus of claim 7, wherein the two illumination sources comprise a first illumination source proximal to the inlet and a second illumination source proximal to the outlet.

9. The apparatus of claim 1, wherein said at least one window comprises two windows.

10. The apparatus of claim 9, wherein the two windows comprise a first window proximal to the inlet, and a second window proximal to the outlet.

11. The apparatus of claim 1, wherein the transparent walls comprise quartz.

12. The apparatus of claim 1, wherein the illumination source comprises ultraviolet illumination light source.

13. The apparatus of claim 1, wherein the liquid comprises water.
